(19) United States
(12) Patent Application Publication
(10) Pub. No.: US 2012/0015056 A1
(43) Pub. Date: Jan. 19, 2012

(54) COMPOSITION COMPRISING EXTRACTS OR FRACTIONS OF SPECIFIC PLANTS, USE THEREOF AND PROCESS FOR PREPARING THE EXTRACTS

(76) Inventor: Gee-Hwoon LEE, Seoul (KR)

(21) Appl. No.: 12/846,289

(22) Filed: Jul. 29, 2010

(30) Foreign Application Priority Data
Jul. 13, 2010 (KR) 10-2010-0067609

(51) Int. Cl. A61K 36/254; A61P 17/14; A61P 35/00

(52) U.S. Cl. 424/728

(57) ABSTRACT

The present invention relates to an extract or fraction of Cinnamomum aromaticum, Arctium lappa, Viticis fructus, Lonicera japonica, Acanthopanax gracilistylis, Raphanus sativus, Astragalus membranaceus and Hordeum vulgare, and a composition comprising said extract or fraction. The invention also relates to the use of said composition for treating or preventing cancers, preventing loss of hair, and stimulating hair growth. The invention also relates to a process for preparing said composition.

### CROSS-REFERENCE TO RELATED APPLICATIONS

This application claims the benefit and priority of Korean Application No. 10˜2010˜0067609, filed Jul. 13, 2010. The entire disclosure of the above application is incorporated herein by reference.

### TECHNICAL FIELD

The present invention relates to an extract or fraction of Cinnamomum aromaticum, Arctium lappa, Viticis fructus, Lonicera japonica, Acanthopanax gracilistylis, Raphanus sativus, Astragalus membranaceus and Hordeum vulgare. The invention also relates to a composition comprising said extract or fraction. The invention also relates to the use of said composition for treating or preventing cancers, preventing loss of hair, and stimulating hair growth. The invention also relates to a process for preparing said composition.

### BACKGROUND ART

Cancer is a devastating and debilitating disease that is becoming more prevalent worldwide. Cancer is distinguished by uncontrolled growth and spread of abnormal cells. It can adversely affect all the organs and tissues of the body, often leading to death. Numerous factors play a role in the initiation and progression of cancer, which makes it difficult to cure. The incidences of cancer among younger individuals have also increased in recent years. About 1,444,000 new cases of cancer were diagnosed in the USA in 2007. This does not include noninvasive cancers at any site except urinary bladder, and does not include basal and squamous cell skin cancer. The molecular diagnosis of cancer relies on biomarker molecules that are antigens or proteins expressed at higher levels in cancer cells than normal cells, or are synthesized de novo. These biomarkers are produced directly by tumor cells or by the human body in response to the presence of cancers. Detection of the biomarkers in a patient's sample can serve as an important step in cancer diagnosis. In addition, the ability to screen cancer at an earlier stage increases the survival rate of cancer patients.

Currently there are many types of antitumor agents utilized for cancer treatments. These agents are classified in several different categories. These categories and examples of the most used antitumor agents are described below.

(1) DNA damaging agents: Doxorubicin is an anthracycline antibiotic that works by intercalating into adjacent nucleotides and blocking RNA and DNA synthesis. To accomplish this, it forms tight DNA-drug interactions and also inhibits topoisomerase II, an enzyme essential for DNA synthesis. Metabolism of Doxorubicin produces free oxygen radicals causing peroxidation of lipid membranes and calcium release from the heart tissues, leading to cardiotoxicity. The major clinical problem in Doxorubicin use is drug resistance. In spite of these side effects, Doxorubicin is used for a wide range of cancers and is the most widely used anthracycline.

(2) Alkylating agents: Cyclophosphamide is the most commonly used alkylating agent. Through cytochrome P-450 action, Cyclophosphamide converts to hydroxylated intermediates and forms active phosphoramide mustard and acrolein. Phosphoramide mustard causes interstrand/intrastrand DNA cross-linkage, causing cell death in wide range of cancer cells. Since Cyclophosphamide is carcinogenic, it increases the risk of developing other cancers and suppresses the immune system.

(3) Microtubule inhibitors (MI): MI disrupt spindle microtubule dynamics and cause cell cycle arrest and apoptosis. Taxanes (paclitaxel and docetaxel) are microtubule polymerizing agents and vinca alkaloids are microtubule depolymerizing agents. Taxanes are the most active agents for treating breast cancer. Paclitaxel binds to β-tubulin, causes microtubule's polymerization and stability, inhibits the metaphase to anaphase transition during mitosis, and induces apoptosis. Docetaxel is a second generation taxane and shares same binding site as paclitaxel with greater affinity. Docetaxel has been shown to have 2 to 4 fold more cytotoxicity than paclitaxel.

(4) Vinca alkaloids: Vincristine, vinblastine, colchicines, podophyllotoxin and nocodazole have high affinity to the ends of microtubules, binding to them and preventing attachment of microtubules to the kinetochores. This causes inhibition of microtubule assembly and destabilizes microtubules leading to apoptosis. They do not share binding sites with taxanes. These MI are generally used as adjuvant therapies to Doxorubicin or Cyclophosphamide treatments.

(5) Aromatase inhibitors (AI): Aromatase coverts androgens into estrogens, thereby increases local estrogen concentrations. This may play a role in breast cancer carcinogenesis. Since Aromatase inhibitors inhibit aromatase but do not block the ovaries from producing estrogen, it only works for post-menopausal women. Aromatase inhibitors can lead to estrogen depletion in the cardiovascular system and bones. Thus, heart problems and osteoporosis are the main side effects.

(6) Non-steroidal hormone therapies: Tamoxifen acts as a selective estrogen receptor modulator (SERM) which exerts antiestrogenic effects by directly binding to the ER α/β and disrupting normal signal transduction in the breast while having estrogenic effects in bone, uterus and cholesterol level, except in patients with ER-negative breast cancers. Its pro-estrogenic effects on the uterus leads to increased chances for development of uterine cancer in breast cancer patients treated with tamoxifen. Raloxifene is next generation of SERM and has anti-estrogenic effects in both breast and uterus. Thus, endometrial growth is not stimulated. Raloxifene was approved by the FDA in 2007 to prevent osteoporosis and risk of invasive breast cancer in postmenopausal women with high risk histories. Raloxifene has pro-estrogen effects in the bone and heart resulting in high density of bones and lowered cholesterol.

All of the above treatment agents have one commonality: they kill cancer and normal cells alike. There are also side effects that greatly decrease the patient's quality of life. There are two promising antitumor agents that have recently been developed. Trastuzumab (Herceptin) is a monoclonal antibody specifically designed to bind to the erbB-2 (her2/neu) receptor. This prevents extracellular growth signals by disrupting ligand and receptor binding, and may induce antibody dependent cellular cytotoxicity. However, Trastuzumab resistance was found at the level of cytoplasmic signal transduction, so additional monoclonal antibodies such as pertuzumab are needed to synergistically block erbB receptor signaling.

Another drug that held great promise was the Tyrosine Kinase Inhibitor GLEEVEC (Imatinib mesilate). Imatinib is a 2-phenylaminopyrimidine derivative that functions as a specific inhibitor of a number of tyrosine kinase enzymes. It functions by occupying the TK active site, leading to a decrease in activity. It is specific for the TK domain in abl (the Abelson protooncogene), c-kit and PDGF-R (platelet-derived growth factor receptor). It works by binding to the ATP binding site of bcr-abl and inhibiting the enzyme activity of the protein competitively. It is selective for bcr-abl, and also inhibits other targets mentioned above (ckit and PDGF-R). However, heart problems, anemia and other side effects in patients treated with GLEEVEC have occurred.

For reasons mentioned above, commercially available antitumor agents have a common problem that if their antitumor effects are enhanced, the resulting high toxicity makes them improper for patients with terminal cancer, the old and children whose body resistance is weak, while if their toxicities are reduced, the desired antitumor effects are not sufficiently obtained. In addition, most of the antitumor agents have side effects such as vomiting, liver toxicity, lung toxicity, neurotoxicity, skin toxicity, hair loss, infertility, and cannot separate between cancer cells and normal cells, and thus they kill cancer cells as well as normal cells. In particular, Paclitaxel, one of the most widely used antitumor agents, is virtually insoluble in water, and thus other substances should be mixed together in order to administer by injection. However, it is reported that the overdose of substances mixed results in cardiotoxicity, hypersensitivity reaction, etc.

Thus, though many ways to kill cancer cells were disclosed in the art, there still remains a need to specifically target cancer cells without toxicities in normal cells, reduce side effects and improve the cytotoxicity of antitumor agents.

Meanwhile, baldness is the state of having no hair or lacking hair where it often grows, especially on the head. When one has faulty hair follicles with no possibility of hair growing out of them and his/her frontal hair line is regressed, he/she is in the state of baldness. Baldness can be divided into two categories—Alopecia areata (circular balding) which is caused by the malfunction of endocrine system and androgenetic alopecia, expression of genetic disposition.

The most common form of baldness is a progressive hair thinning condition called androgenic alopecia or “male pattern baldness” that occurs in adult male humans and other species. There are two drugs, Finasteride and Minoxidil, that have been approved by the FDA. However, these drugs can merely retard the progression of hair loss, but do not solve the root cause of hair loss. In addition, hair loss may cause the person to limit social activities, and surveys have shown that around 40% of women with alopecia have had marital problems. In extreme circumstances, some people really take hair loss badly and get highly distressed about it, up to the point of getting into depression. Furthermore, a hair transplant surgery is too expensive so that it is hard for the public to undergo it. Thus, there remains a need in the art to fundamentally treat or prevent hair loss.

### DISCLOSURE

### Technical Problem

It is the object of the present invention to provide a pharmaceutical composition usable to target only abnormal cells such as cancer cells, activate the function of normal cells, repair injured normal cells, have no toxicity, apply to patients with terminal cancer whose body resistance is weak, prepare under the mild condition, have a hydrophile property which makes it much safer in production than that of using organic solvents and available in both injection and oral administration form, and work fast.

The invention also relates to the composition usable to fundamentally treat or prevent hair loss.

### Technical Solution

The present invention relates to an extract or fraction of Cinnamomum aromaticum, Arctium lappa, Viticis fructus, Lonicera japonica, Acanthopanax gracilistylis, Raphanus sativus, Astragalus membranaceus and Hordeum vulgare, and a composition comprising said extract or fraction.

The invention also relates to the use of said composition for treating or preventing cancers. Such cancers can include, but are not limited to, breast cancer, prostate cancer, lung cancer, liver cancer, pancreatic cancer, skin cancer, colorectal cancer, osteosarcoma, brain tumor, etc. The composition according to the invention is effective against all kinds of cancers.

The invention also relates to the use of said composition for preventing loss of hair or stimulating hair growth.

The invention also relates to a process for preparing the composition comprising the extracts of the invention, including:
a) placing Cinnamomum aromaticum, Arctium lappa, Viticis fructus, Lonicera japonica, Acanthopanax gracilistylis, Raphanus sativus, Astragalus membranaceus and Hordeum vulgare in an extraction apparatus with water, and
b) heating said extraction apparatus to obtain the extracts.

In accordance with one embodiment, water used in the step a) is the water purified by reverse-osmosis membrane filter system.

In accordance with one embodiment, the heating in the step b) is performed at temperature of 100˜120° C. for 4˜5 hours.

In accordance with one embodiment, the extracts obtained in the step b) are filtered by water at 100˜150° C., preferably are then filtered by water at 70˜100° C., more preferably are then filtered by water at 40˜60° C., even more preferably are then filtered by water at 15˜30° C., and most preferably are then filtered by water at −1˜15° C.

Preferably, the filter used in said filtering is the membrane with a pore size of not bigger than 10⁻⁶ m.

The composition comprising extracts or fractions of the invention may be administered by parenteral (e.g. subcutaneously, intramuscularly, intravenously, intraperitoneally, intrapleurally, intravesicularly or intrathecally), topical, oral, rectal, nasal route, etc. And, the dose and duration of the treatment will depend on a variety of factors, including the age, body weight, general health, sex, diet and the disease type of the patient.

It is generally known in the art that Cinnamomum aromaticum can be used to reduce a fever, control the body's temperature, increase absorption of drugs, or improve effects of drugs. Said extract or fraction of Cinnamomum aromaticum comprises 2-methyl-butyric acid, etc. (see Dr. Duke's Phytochemical and Ethnobotanical Databases [Online Database]).

It is generally known in the art that Arctium lappa can be used to ensure smooth neurotransmissions in the nervous system, help the function of spleen, or stimulate the body's secretion of hormones. Said extract or fraction of Arctium lappa comprises arctigenin-4-O-glucoside, 3,5,7,9-tetrayne, alanine, etc. (see Dr. Duke's Phytochemical and Ethnobotanical Databases [Online Database]).

It is generally known in the art that Viticis fructus can be used to produce lymph, activate the function of lymph, improve the function of heart, or purify the blood. Said extract or fraction of Viticis fructus comprises vitexicarpin, hesperidin, etc. (see Sam Sik Kang et al., Phytochemical Analysis of Viticis Fructus, Kor. J. Pharmacogn 25(3): 214˜220 (1994)).

It is generally known in the art that Lonicera japonica stimulates hormonal regulations, act on hypothalamus, and stimulates the motor center. Said extract or fraction of Lonicera japonica comprises luteolin-7-rhamnoglucoside, saponin, pinene, trans-geraniol, linalol, chlorogenic acid, etc. (see Dr. Duke's Phytochemical and Ethnobotanical Databases [Online Database]).

It is generally known in the art that Acanthopanax gracilistylis can be used to treat orchitis or inflammation of the throat, or activate T-helper cells. Said extract or fraction of Acanthopanax gracilistylis comprises copper, etc. (see Dr. Duke's Phytochemical and Ethnobotanical Databases [Online Database]).

It is generally known in the art that Raphanus sativus can be used to build up cardiovascular strength, or stimulate cells to repair the adventitia of nervous system. Said extract or fraction of Raphanus sativus comprises sinigrin, etc. (see Dr. Duke's Phytochemical and Ethnobotanical Databases [Online Database]).

It is generally known in the art that Astragalus membranaceus can be used to inhibit the excessive acidification of gastric juices in the gut, and thereby recover the nervous system. Said extract or fraction of Astragalus membranaceus comprises 3′-hydroxyformononetin, kaempferol, water, etc. (see Dr. Duke's Phytochemical and Ethnobotanical Databases [Online Database]).

It is generally known in the art that Hordeum vulgare helps the gut function and can be used when the secretion of gastric juices is reduced or the gut is not in good condition. Said extract or fraction of Hordeum vulgare comprises valine, etc. (see Dr. Duke's Phytochemical and Ethnobotanical Databases [Online Database]).

When the composition according to the invention is administered into the body, it is found that neurotransmitters are activated, the function of spleen is enhanced, and the blood is purified. Thereby, the composition according to the invention can stimulate the secretion of neurotransmitters and hormones in the body, increase the activity of spleen, and reduce a fever, thus controlling the body's temperature. In addition, it can activate the overall function of lymph, proliferate lymphocytes and macrophagocytes, thus purifying lymph and blood. Furthermore, while most cancer patients can not tolerate conventional antitumor agents having strong toxicity because of weakened body functions and complications, the composition according to the invention can activate the function of normal cells and repair injured normal cells, thereby enhance the weakened body functions of patients, and provide the patient's overall physical condition suitable to be treated with anticancer treatments. Therefore, the composition according to the invention can be used to patients with terminal cancer without causing side effects and drug-shock. And, when the composition according to the invention is administered into the body, it is found that cancer cells stop temporarily their activities, and then the form of cancer cells changes to the polygonal form with black edges, and eventually bursts. As well, the composition according to the invention stimulates marrow, and produces NK and NKT cells. It is differentiated into T and B cells, eliminating cancer cells that blood can reach.

### Advantageous Effects

The composition according to the invention is effective in treating and preventing all kinds of cancers. In particular, unlike conventional antitumor agents to kill both cancer and normal cells, the composition according to the invention can target only abnormal cells such as cancer cells, activate the function of normal cells, and repair injured normal cells. In addition, the composition works very fast and recovers the body's functioning weakened by cancer and complications, and thus is suitable for patients with terminal cancer as well as the old and children. Furthermore, the composition can eliminate latent tumors in the body and enhance immunocyte activity in the blood, thereby prevent cancer in advance.

Moreover, the composition according to the invention can prevent loss of hair and stimulate hair growth, resulting in cost-saving on hair transplant surgery.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1 and 2 are the graphs showing the cell counts determined in cell line HCC 1419 during the 1^{st} week (LC50) and the 2^{nd} week (recovery), respectively. Herein, the horizontal axis represents the time in culture, and the vertical axis represents 1/20 of cell number.

FIGS. 3 and 4 are the graphs showing the viability determined in cell line HCC 1419 by XTT assay at a wavelength of 500 nm during the 1^{st} week (LC50) and the 2^{nd} week (recovery), respectively. Herein, the horizontal axis represents the time in culture, and the vertical axis represents 1/100 of viability (%).

FIGS. 5 and 6 are the graphs showing the cytotoxicity determined in cell line HCC 1419 by XTT assay at a wavelength of 500 nm during the 1^{st} week (LC50) and the 2^{nd} week (recovery), respectively. Herein, the horizontal axis represents the time in culture, and the vertical axis represents 1/100 of cytotoxicity (%).

FIG. 7 is the graph showing the cell counts determined in cell line HME during the 1^{st} week (LC50). Herein, the horizontal axis represents the time in culture, and the vertical axis represents 1/20 of cell number.

FIG. 8 is the graph showing the cell counts determined in cell line BJ during the 1^{st} week (LC50). Herein, the horizontal axis represents the time in culture, and the vertical axis represents 1/20 of cell number.

FIG. 9 is the graph showing the viability determined in cell line MDA-MB-231 by XTT assay at a wavelength of 450 nm. Herein, the horizontal axis represents the time points 12, 84, 108 and 132 hours after treatment, and the vertical axis represents the value of [the treated group−the blank group].

FIG. 10 is the graph showing the viability determined in cell line TTU-1 by XTT assay at a wavelength of 450 nm. Herein, the horizontal axis represents the time points 12, 84, 108 and 132 hours after treatment, and the vertical axis represents the value of [the treated group−the blank group].

FIG. 11 is the graph showing the viability determined in cell line A549 by MTT assay at a wavelength of 500 nm. Herein, the horizontal axis represents the time points 24, 48, 72 and 96 hours after treatment, and the vertical axis represents the value of the treated group.

FIG. 12 is the graph showing the viability determined in cell line HepG2 by MTT assay at a wavelength of 500 nm. Herein, the horizontal axis represents the time points 24, 48, 72 and 96 hours after treatment, and the vertical axis represents the value of the treated group.

FIGS. 13A-D, 14A-D and 15A-D are the photographs taken at 24, 48 and 72 hours after treatment of cell line HCT-15 with the composition according to the invention or PBS, respectively. Herein, each of the top photographs represent the group treated with the composition according to the invention and then H&E-stained, while each of the bottom photographs represent the group treated with PBS and then H&E-stained.

FIG. 16 is the photograph taken at 14 days after the composition according to the invention was administered to 5-week-old female nude mice (BALB/c nu/nu).

FIG. 17 is the photograph taken at 21 days after the composition according to the invention was administered to 5-week-old female nude mice (BALB/c nu/nu).

### EXAMPLES

The present invention is described in further detail in the following Examples which are not in any way intended to limit the scope of the invention as claimed. In addition, it will appear to those ordinarily skilled in the art that various modifications may be made to the disclosed embodiments, and that such modifications are intended to be within the scope of the present invention.

### Example 1

Cinnamomum aromaticum, Arctium lappa, Viticis fructus, Lonicera japonica, Acanthopanax gracilistylis, Raphanus sativus, Astragalus membranaceus and Hordeum vulgare were weighed by an electronic scale. And then, 4 g of Cinnamomum aromaticum, 2 g of Arctium lappa, 4 g of Viticis fructus, 2 g of Lonicera japonica, 2 g of Acanthopanax gracilistylis, 2 g of Raphanus sativus, 2 g of Astragalus membranaceus and 4 g of Hordeum vulgare was placed in a spunbond nonwoven fabric, and the nonwoven fabric was sealed, and then fixed within an extraction apparatus with 180 L of water. Said water is the water purified by reverse-osmosis membrane filter system. Then, the extraction apparatus was heated at 100˜110° C., and it takes about one hour until water boils. After water boils, the extraction apparatus was additionally heated for 4 hours.

Some of vapor was discharged through the outlet on top of the extraction apparatus to let go of arbitrary toxic substances of herbs, while some remained and was cooled through a cooler and reinjected to the extraction apparatus.

After the additional heating for 4 hours was complete, the obtained extracts were successionally filtered by water at 100˜150° C., water at 70˜100° C., water at 40˜60° C., water at 15˜30° C., and water at −1˜15° C.

### Example 2

The procedure of Example 1 for obtaining extracts was followed with the exception that 4 g of Cinnamomum aromaticum, 2 g of Arctium lappa, 4 g of Viticis fructus, 4 g of Lonicera japonica, 2 g of Acanthopanax gracilistylis, 2 g of Raphanus sativus, 2 g of Astragalus membranaceus and 4 g of Hordeum vulgare were used.

### Example 3

The procedure of Example 1 for obtaining extracts was followed with the exception that the extraction apparatus was heated at 110˜120° C. and was further heated for 5 hours after water boiled.

### Example 4

Cancer cell line HCC 1419 and normal cell lines HME 50 HT and BJ were tested for changes in metabolism (XTT assay) and cell death (cell counts). Each of the cell lines was cultured in the medium shown in the following Table 1.

TABLE 1

| Cell lines | Medium formulation |
|---|---|
| HCC 1419 | DMEM plus 10% fetal bovine serum |
| HME 50 HT | Serum-free medium (SF-171 from Clonetics Corp., San |
| | Diego, Calif.) |
| BJ | DMEM plus 10% fetal bovine serum |

* DMEM = Dulbecco's Minimum Essential Medium

Cells were plated into 48-well Corning CellBind™ plates at densities of 20,000 cells for normal cells and 10,000˜20,000 cells for cancer cells. 24 to 48 h after plating, cells were treated with the extracts obtained in the Examples 1 to 3 diluted by 20 times with purified water, or Taxol. The first time point treated with the composition of the invention is 0 h. At 3 days after the first treatment with the composition of the invention, medium was changed and the composition of the invention at the same concentrations was added to the cultured cells.

Concurrently, Taxol at concentrations of 2.2e-7M for 24 h were added to parallel cultures of cells 24˜48 h after plating as the positive control group, whereas medium alone was used as the negative control group.

### Cell Counts:

Cells were harvested using Trypsin-EDTA at the designated time points. Total cells for each well were counted with a Beckman-Coulter Z1 Particle Characterization Unit. Two 48-well plates were used per cell line, and they were measured at 0 h, 12 h, 24 h, 48 h, 4 d and 7 d, respectively.

In the 1^{st} plate, cells were treated with the composition according to the invention and medium was changed at 0 h and 3 d. Cells treated with Taxol were exposed for 24 h, and then medium was replaced with untreated medium. As a result, LC-50 curves were generated in the 1^{st} week.

In the 2^{nd} plate, 7 d of the 1^{st} week (LC50) is the same as 0 h of the 2^{nd} week (recovery).

At 0 h of the 2^{nd} week, medium was exchanged with untreated medium for the remainder of the experiment. The untreated controls were not measured in the 2^{nd} week (recovery) due to overconfluence by the end of the 1^{st} week. As a result, Recovery curves were generated in the 2^{nd} week.

(1) HCC 1419

HCC 1419 is a primary ductal carcinoma cell line. The cells are poorly differentiated, overexpressing Her2-neu, negative for p53 expression. HCC1419 is positive for the epithelial cell specific marker, epithelial glycoprotein 2 (EGP2) and for cytokeratin 19. The cells are negative for estrogen receptor and progesterone receptor.

The XTT Cell Viability Assay is an accepted analysis technique for viability and cytotoxicity of anticancer drugs or other pharmaceutical compositions. Cells were seeded in a 96-well tissue culture plate at a density of 10,000 cells for normal cells and 5,000 cells for cancer cells. After incubation period, the formazan dye formed was quantitated using an ELISA reader. The optimal wavelength used in experiments was 500 nm, and it was measured at 0 h, 12 h, 24 h, 48 h, 4 d and 7 d, respectively. The obtained values were generated using the software SoftMax Pro 4.8.

For determining viability and cytotoxicity by XTT assay, the cell viability was calculated according to the manufacturer's instructions as follows:

[Treated(the composition of the invention or Taxol)−blank]/Control(untreated)×100%

Cellular cytotoxicity was calculated according to the manufacturer's instructions as follows (the term “blank” means XTT in medium only):

[Control(untreated)−(Treated(the composition of the invention or Taxol)−blank)]/Control(untreated)×100%

The experimental group treated with the composition of the invention, the control group treated with Taxol, and the untreated group were treated and measured in the same manner.

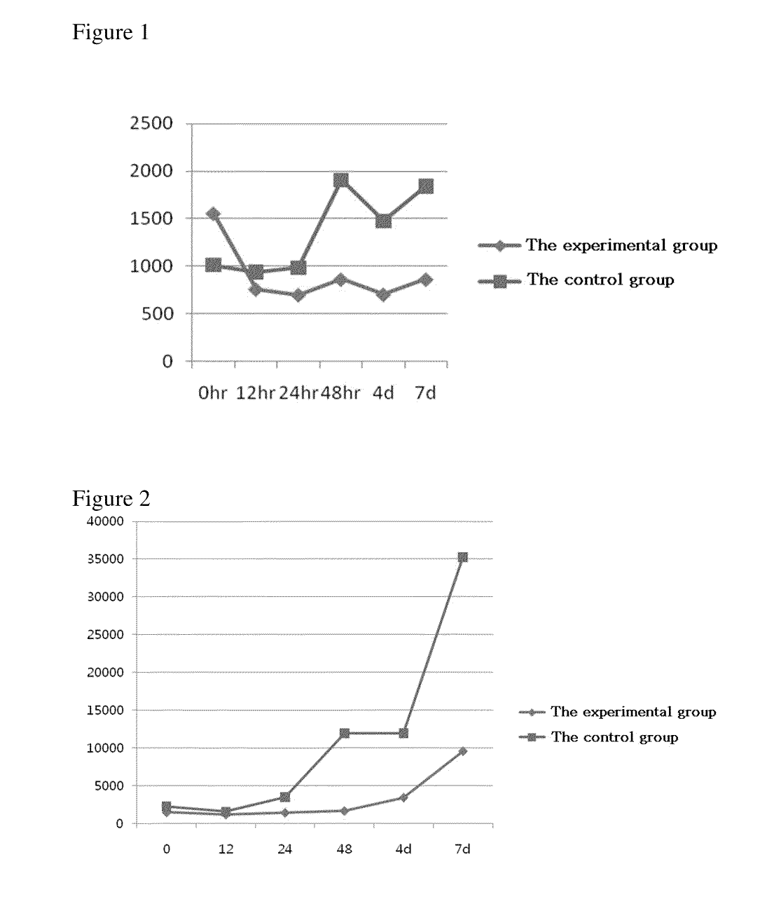

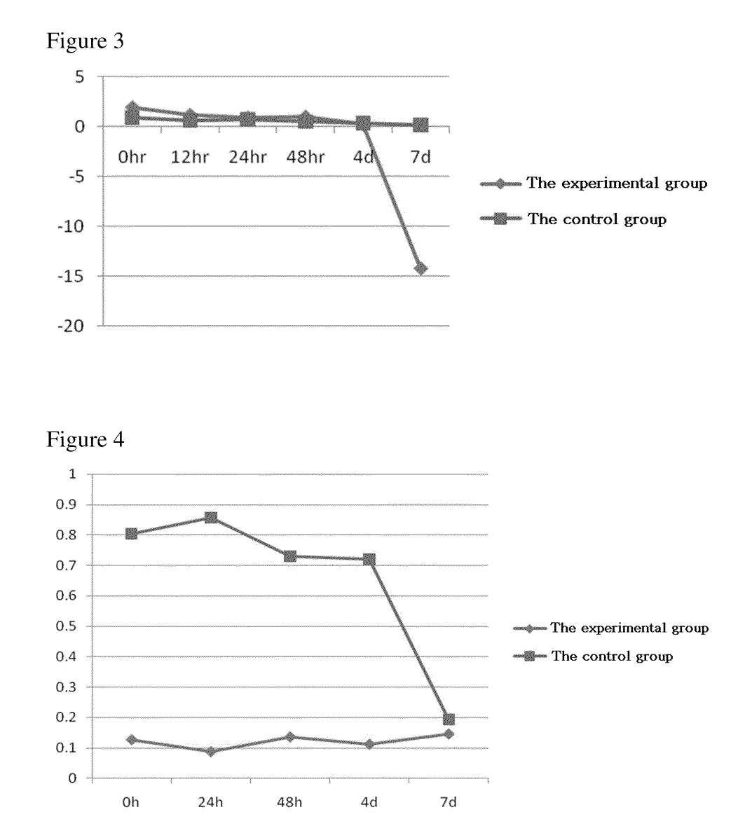

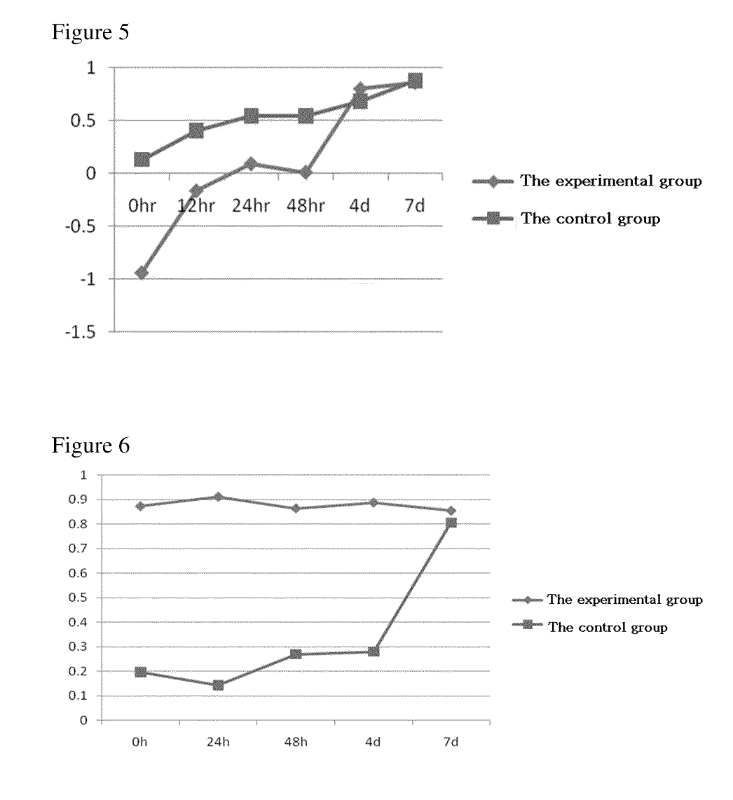

FIGS. 1 and 2 shows the graph representing the cell counts determined in cell line HCC 1419 during the 1st week (LC50) and the 2^{nd} week (recovery), respectively, wherein the horizontal axis represents the time in culture, and the vertical axis represents 1/20 of cell number. FIGS. 3 and 4 shows the graph representing the viability determined in cell line HCC 1419 by XTT assay at a wavelength of 500 nm during the 1^{st} week (LC50) and the 2^{nd} week (recovery), respectively, wherein the horizontal axis represents the time in culture, and the vertical axis represents 1/100 of viability (%). FIGS. 5 and 6 shows the graph representing the cytotoxicity determined in cell line HCC 1419 by XTT assay at a wavelength of 500 nm during the 1^{st} week (LC50) and the 2^{nd} week (recovery), respectively, wherein the horizontal axis represents the time in culture, and the vertical axis represents 1/100 of cytotoxicity (%).

(2) HME 50 HT—Normal Epithelial Cells

Human Mammary Epithelial (HME) cells were derived from adjacent normal tissue.

During the experiment, no cytotoxicity was observed in the group treated with the composition according to the invention. Surprisingly, the HME 50 HT cells thrived and became over confluent during recovery. And, it was found from the photomicrographs that the cells appeared very healthy and unstressed.

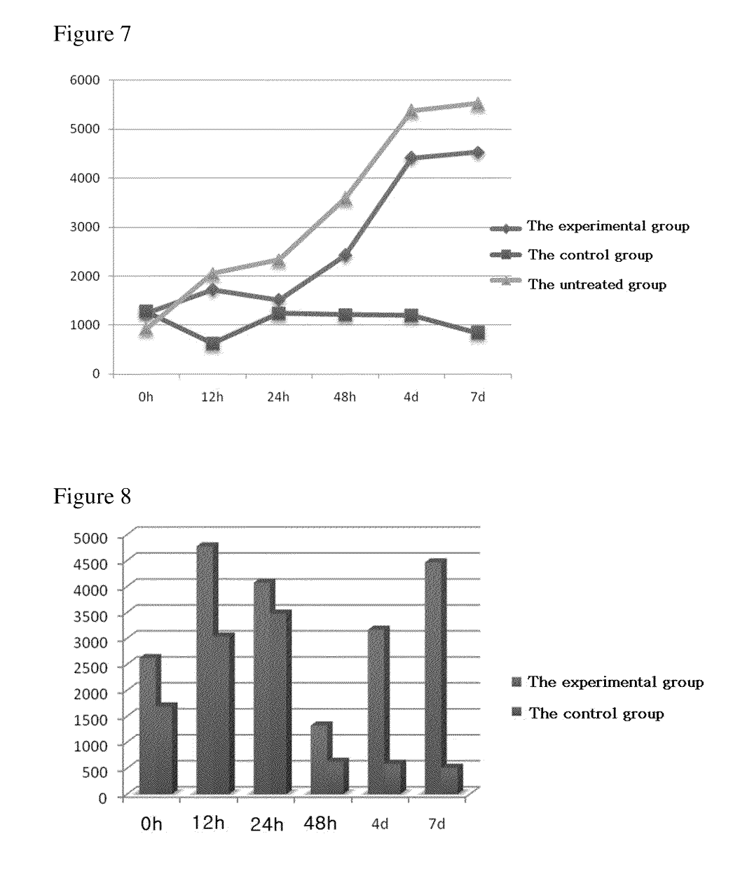

FIG. 7 shows the graph representing the cell counts determined in cell line HME during the 1^{st} week (LC50), wherein the horizontal axis represents the time in culture, and the vertical axis represents 1/20 of cell number.

(3) BJ—Normal Fibroblasts

BJ cells were derived from normal foreskin of a newborn. Although they have the capacity to proliferate to a maximum of 72 population doublings before the onset of senescence, they are negative for telomerase.

The group treated with the composition according to the invention showed no toxicity, and the cells were very healthy and unstressed. Also, enhanced cell growth was observed. FIG. 8 shows the graph representing the cell counts determined in cell line BJ during the 1^{st} week (LC50), wherein the horizontal axis represents the time in culture, and the vertical axis represents 1/20 of cell number.

### Example 5

Cancer cell line HCC 1419 and normal cell lines HME 50 HT and BJ were tested for cellular cytotoxicity, and XTT assay, MTT assay and/or photography was performed.

1. Cell Lines and Analysis Methods

TABLE 2

| Cell lines | Analysis methods |
|---|---|
| MDA-MB-231 (breast cancer), TTU-1 (breast cancer) | XTT assay and |
| | photography |
| A549 (lung cancer), HepG2 (liver cancer) | MTT assay |
| HCT-15(colorectal cancer) | photography |

2. Experiment Method

(1) XTT Assay

Each of 5×103 breast cancer cells MDA-MB-231 and TTU-1 were seeded in a 96-well flat-bottomed microplate, and cultured in CO2 incubator. Cells were seeded in a 96-well tissue culture plate at a density of 10,000 cells for normal cells and 5,000 cells for cancer cells. DMEM plus 10% fetal bovine serum was used as a culture medium. At 24 hours after seeding, the extracts obtained in the Examples 1 to 3 were added to each well. At 12 hours after added the extracts, XTT solutions were added and then the cells were incubated for 2 hours. After that, absorbance value were measured using microplate spectrophotometer at a wavelength of 450 nm. In addition, Annexin-V/PI-stained MDA-MB-231 and TTU-1 cells were photographed and observed.

(2) MTT Assay

Similarly, MTT assay was performed for lung cancer cell line A549 and liver cancer cell line HepG2. But, MTT solutions were added instead of XTT solutions; as a culture medium, RPMI1640 with L-glutamine (300 mg/L), 25 mM HEPES and 25 mM NaHCO3 90% and heat-inactivated fetal bovine serum (FBS) 10% was used for A549, and minimum essential medium, 25 mM HEPES and 25 mM NaHCO3 90% and heat-inactivated fetal bovine serum (FBS) 10% was used for HepG2; and MTT values were measured at 1 d, 2 d, 3 d and 4 d, respectively.

(3) Cell Treatments

Colorectal cancer cell line HCT-15 was put into the round bottom flask. As a culture medium, RPMI1640 with L-glutamine (300 mg/L), 25 mM HEPES and 25 mM NaHCO3 90% and heat-inactivated fetal bovine serum (FBS) 10% were used. After the cells were sufficiently incubated, they were treated with the extracts obtained in the Examples 1 to 3. And then, at 8 h, 24 h, 48 h and 72 h, H&E-stained HCT-15 cells were photographed and observed.

3. Treatment Methods

TABLE 3

| | Concentration used in | |
|---|---|---|
| Cell lines | the experimental group | Concentration used in the control group |
| MDA-MB- | The composition of the | Control group 1. Medium 100% |
| 231, TTU-1 | invention: Medium = | Control group 2. PBS: Medium = 5:5, 6:4, 7:3, 8:2 |
| | 5:5, 6:4, 7:3, 8:2 | Control group 3. Taxol 1.2 × 10⁻⁷ M (concentration |
| | | in plasma at 24 hours after administration) |
| | | Control group 4. Taxol 2.5 × 10⁻⁶ M (concentration |
| | | in plasma at 3 hours after administration) |
| A549, | The composition of the | Control group 1. Medium 100% |
| HepG2 | invention: Medium = | Control group 2. PBS: Medium = 5:5, 6:4, 7:3, 8:2 |
| | 5:5, 6:4, 7:3, 8:2 | Control group 3. Taxol 35.1 uM/ml |
| | | Control group 4. Taxol 70.2 uM/ml |
| | | Control group 5. 5-FU 1,000 ppm |
| HCT-15 | The composition of the | Control group 1. Medium 100% |
| | invention: Medium = | Control group 2. PBS: Medium = 7:3 |
| | 7:3 | |

4. The Results

In the following, the values for the composition of the invention indicate the mean value of values obtained using the extracts of the Examples 1 to 3.

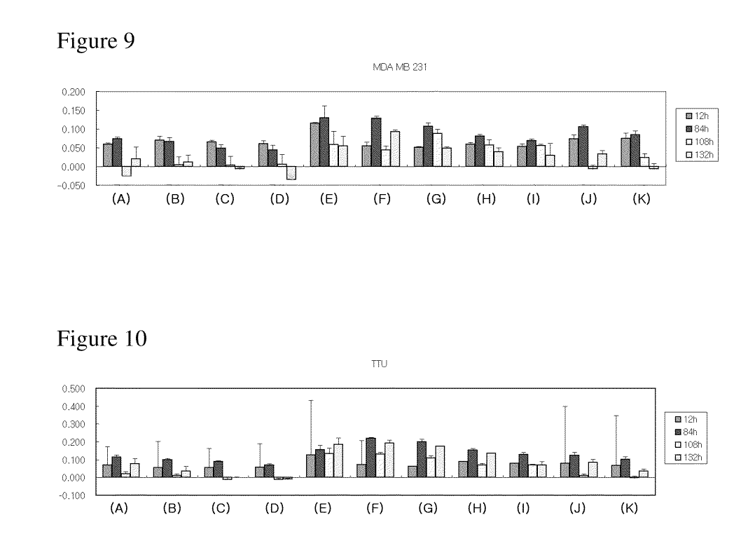

The following Table 4 and FIG. 9 show the value of [the treated group−the blank group] determined in cell line MDA-MB-231.

TABLE 4

| | Mean value | | | | Standard deviation | | | |
|---|---|---|---|---|---|---|---|---|
| | 12 h | 84 h | 108 h | 132 h | 12 h | 84 h | 108 h | 132 h |
| (A) The composition | 0.059 | 0.073 | −0.026 | 0.021 | 0.004 | 0.005 | 0.015 | 0.032 |
| of the invention: | | | | | | | | |
| Medium = 5:5 | | | | | | | | |
| (B) The composition | 0.070 | 0.066 | 0.005 | 0.012 | 0.011 | 0.011 | 0.021 | 0.019 |
| of the invention: | | | | | | | | |
| Medium = 6:4 | | | | | | | | |
| (C) The composition | 0.065 | 0.049 | 0.003 | −0.006 | 0.005 | 0.010 | 0.025 | 0.003 |
| of the invention : | | | | | | | | |
| Medium = 7:3 | | | | | | | | |
| (D) The composition | 0.061 | 0.044 | 0.006 | −0.034 | 0.009 | 0.014 | 0.027 | 0.008 |
| of the invention : | | | | | | | | |
| Medium = 8:2 | | | | | | | | |
| (E) Medium 100% | 0.115 | 0.130 | 0.059 | 0.055 | 0.003 | 0.033 | 0.035 | 0.026 |
| (F) PBS: Medium = 5:5 | 0.055 | 0.128 | 0.044 | 0.092 | 0.010 | 0.007 | 0.010 | 0.005 |
| (G) PBS: Medium = 6:4 | 0.051 | 0.107 | 0.088 | 0.049 | 0.003 | 0.010 | 0.012 | 0.003 |
| (H) PBS: Medium = 7:3 | 0.059 | 0.081 | 0.057 | 0.039 | 0.005 | 0.005 | 0.014 | 0.011 |
| (I) PBS: Medium = 8:2 | 0.053 | 0.069 | 0.056 | 0.029 | 0.007 | 0.004 | 0.003 | 0.032 |
| (J) Taxol 1.2 × 10⁻⁷M | 0.074 | 0.107 | −0.006 | 0.034 | 0.010 | 0.004 | 0.010 | 0.009 |
| (K) Taxol 2.5 × 10⁻⁶M | 0.076 | 0.085 | 0.024 | −0.007 | 0.014 | 0.010 | 0.011 | 0.014 |

The following Table 5 and FIG. 10 show the value of [the treated group−the blank group] determined in cell line TTU-1.

TABLE 5

| | Mean value | | | | Standard Deviation | | | |
|---|---|---|---|---|---|---|---|---|
| | 12 h | 84 h | 108 h | 132 h | 12 h | 84 h | 108 h | 132 h |
| (A) The composition | 0.069 | 0.113 | 0.021 | 0.078 | 0.014 | 0.014 | 0.012 | 0.030 |
| of the invention: | | | | | | | | |
| Medium = 5:5 | | | | | | | | |
| (B) The composition | 0.054 | 0.099 | 0.011 | 0.036 | 0.008 | 0.008 | 0.011 | 0.025 |
| of the invention: | | | | | | | | |
| Medium = 6:4 | | | | | | | | |
| (C) The composition | 0.055 | 0.088 | −0.011 | 0.001 | 0.005 | 0.005 | 0.012 | 0.003 |
| of the invention: | | | | | | | | |
| Medium = 7:3 | | | | | | | | |
| (D) The composition | 0.057 | 0.069 | −0.012 | −0.009 | 0.011 | 0.007 | 0.007 | 0.004 |
| of the invention: | | | | | | | | |
| Medium = 8:2 | | | | | | | | |
| (E) Medium 100% | 0.127 | 0.155 | 0.134 | 0.186 | 0.013 | 0.025 | 0.033 | 0.037 |
| (F) PBS: Medium = 5:5 | 0.071 | 0.219 | 0.130 | 0.193 | 0.004 | 0.006 | 0.012 | 0.017 |
| (G) PBS: Medium = 6:4 | 0.062 | 0.199 | 0.108 | 0.175 | 0.001 | 0.015 | 0.014 | 0.004 |
| (H) PBS: Medium = 7:3 | 0.090 | 0.154 | 0.070 | 0.136 | 0.018 | 0.009 | 0.010 | 0.004 |
| (I) PBS: Medium = 8:2 | 0.079 | 0.128 | 0.070 | 0.071 | 0.014 | 0.013 | 0.006 | 0.018 |
| (J) Taxol 1.2 × 10⁻⁷M | 0.079 | 0.123 | 0.012 | 0.084 | 0.010 | 0.018 | 0.008 | 0.018 |
| (K) Taxol 2.5 × 10⁻⁶M | 0.067 | 0.101 | −0.002 | 0.035 | 0.009 | 0.016 | 0.009 | 0.013 |

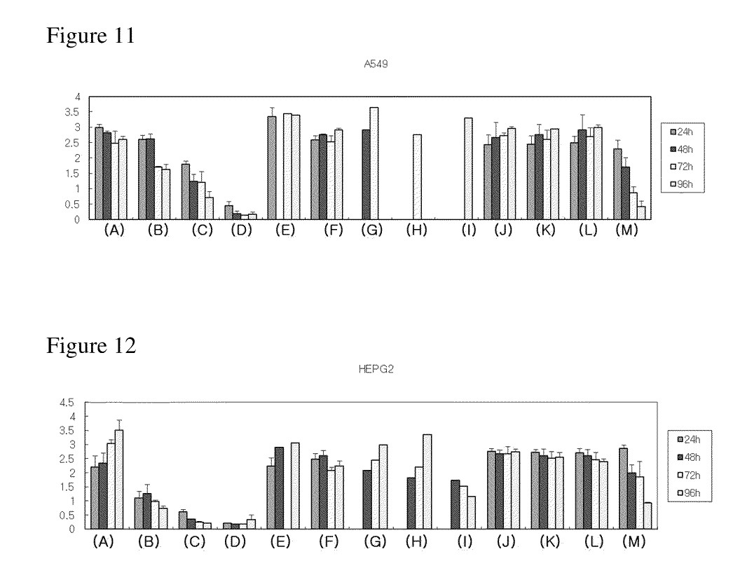

The following Table 6 and FIG. 11 show the value of the treated group determined in cell line A549.

TABLE 6

| | Mean value | | | | Standard Deviation | | | |
|---|---|---|---|---|---|---|---|---|
| | 24 h | 48 h | 72 h | 96 h | 24 h | 48 h | 72 h | 96 h |
| (A) The composition | 2.988 | 2.816 | 2.474 | 2.595 | 0.104 | 0.07 | 0.403 | 0.108 |
| of the invention: | | | | | | | | |
| Medium = 5:5 | | | | | | | | |
| (B) The composition | 2.597 | 2.613 | 1.698 | 1.627 | 0.148 | 0.176 | 0.038 | 0.167 |
| of the invention: | | | | | | | | |
| Medium = 6:4 | | | | | | | | |
| (C) The composition | 1.802 | 1.24 | 1.202 | 0.707 | 0.108 | 0.227 | 0.362 | 0.209 |
| of the invention: | | | | | | | | |
| Medium = 7:3 | | | | | | | | |
| (D) The composition | 0.449 | 0.189 | 0.131 | 0.175 | 0.132 | 0.082 | 0.023 | 0.079 |
| of the invention: | | | | | | | | |
| Medium = 8:2 | | | | | | | | |
| (E) Medium 100% | 3.337 | 0.6813 | 3.4414 | 3.3868 | 0.306 | | | |
| (medium was changed | | | | | | | | |
| at 24 h) | | | | | | | | |
| (F) PBS: Medium = 5:5 | 2.588 | 2.759 | 2.525 | 2.905 | 0.135 | 0.02 | 0.194 | 0.065 |
| (G) PBS: Medium = 6:4 | | 2.9105 | 3.639 | | | | | |
| (H) PBS: Medium = 7:3 | | >4 | 2.7511 | | | | | |
| (I) PBS : Medium = 8:2 | | >4 | >4 | 3.2984 | | | | |
| (J) Medium 100% | 2.427 | 2.663 | 2.721 | 2.961 | 0.321 | 0.49 | 0.095 | 0.063 |
| (medium was not | | | | | | | | |
| changed) | | | | | | | | |
| (K) Taxol 35.1 uM/ml | 2.438 | 2.753 | 2.6 | 2.934 | 0.281 | 0.348 | 0.307 | 0.024 |
| (L) Taxol 70.2 uM/ml | 2.488 | 2.911 | 2.691 | 2.98 | 0.223 | 0.491 | 0.299 | 0.094 |
| (M) 5-FU 1,000 ppm | 2.298 | 1.697 | 0.863 | 0.42 | 0.281 | 0.314 | 0.211 | 0.184 |

The following Table 7 and FIG. 12 show the value of the treated group determined in cell line HepG2.

TABLE 7

| | Mean value | | | | Standard Deviation | | | |
|---|---|---|---|---|---|---|---|---|
| | 24 h | 48 h | 72 h | 96 h | 24 h | 48 h | 72 h | 96 h |
| (A) The composition | 2.201 | 2.333 | 3.033 | 3.507 | 0.404 | 0.366 | 0.138 | 0.362 |
| of the invention: | | | | | | | | |
| Medium = 5:5 | | | | | | | | |
| (B) The composition | 1.112 | 1.267 | 0.982 | 0.745 | 0.232 | 0.329 | 0.058 | 0.085 |
| of the invention: | | | | | | | | |
| Medium = 6:4 | | | | | | | | |
| (C) The composition | 0.611 | 0.356 | 0.25 | 0.214 | 0.089 | 0.015 | 0.027 | 0.018 |
| of the invention: | | | | | | | | |
| Medium = 7:3 | | | | | | | | |
| (D) The composition | 0.214 | 0.174 | 0.18 | 0.334 | 0.011 | 0.03 | 0.007 | 0.182 |
| of the invention: | | | | | | | | |
| Medium =8:2 | | | | | | | | |
| (E) Medium 100% | 2.229 | 2.8975 | >4 | 3.0531 | 0.298 | | | |
| (medium was | | | | | | | | |
| changed at 24 h) | | | | | | | | |
| (F) PBS: Medium = 5:5 | 2.471 | 2.607 | 2.086 | 2.231 | 0.211 | 0.192 | 0.12 | 0.195 |
| (G) PBS: Medium = 6:4 | | 2.074 | 2.4457 | 2.9809 | | | | |
| (H) PBS: Medium = 7:3 | | 1.8121 | 2.2046 | 3.3468 | | | | |
| (I) PBS: Medium = 8:2 | | 1.7337 | 1.5252 | 1.1556 | | | | |
| (J) Medium 100% | 2.761 | 2.669 | 2.662 | 2.735 | 0.101 | 0.14 | 0.263 | 0.111 |
| (medium was not | | | | | | | | |
| changed) | | | | | | | | |
| (K) Taxol 35.1 uM/ml | 2.73 | 2.604 | 2.506 | 2.546 | 0.098 | 0.236 | 0.244 | 0.179 |
| (L) Taxol 70.2 uM/ml | 2.71 | 2.595 | 2.466 | 2.389 | 0.156 | 0.224 | 0.249 | 0.112 |
| (M) 5-FU 1,000 ppm | 2.867 | 1.998 | 1.853 | 0.938 | 0.111 | 0.292 | 0.553 | 0.033 |

As seen in the FIGS. 9 to 12, the value for the experimental group treated with the composition of the invention is approaching to 0, as time goes by, while the value for the control group treated with PBS is increasing as well as the control group treated with Taxol. This shows that the cancer cells were almost killed in the experimental group.

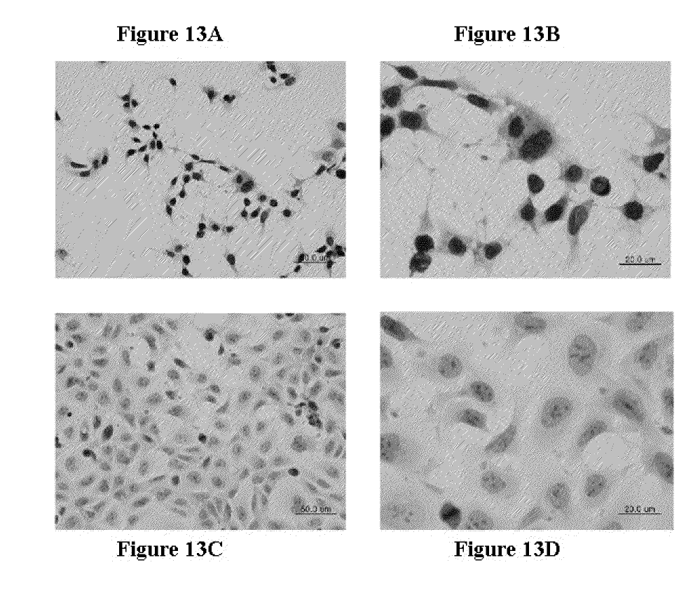

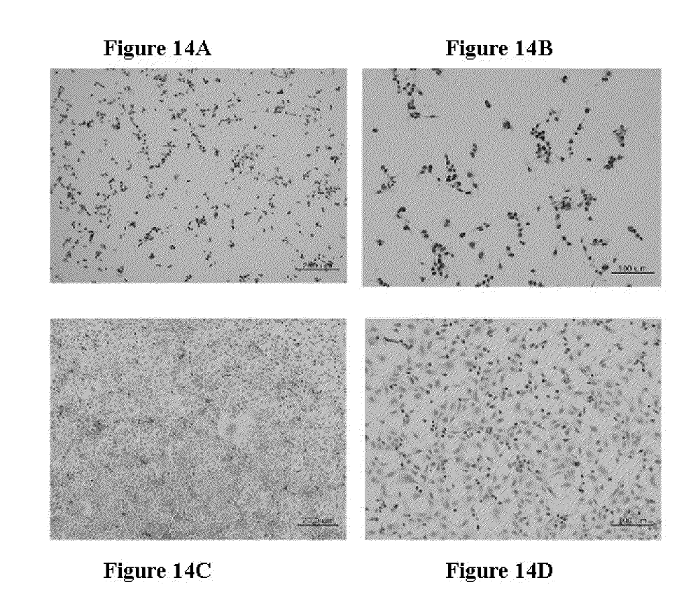

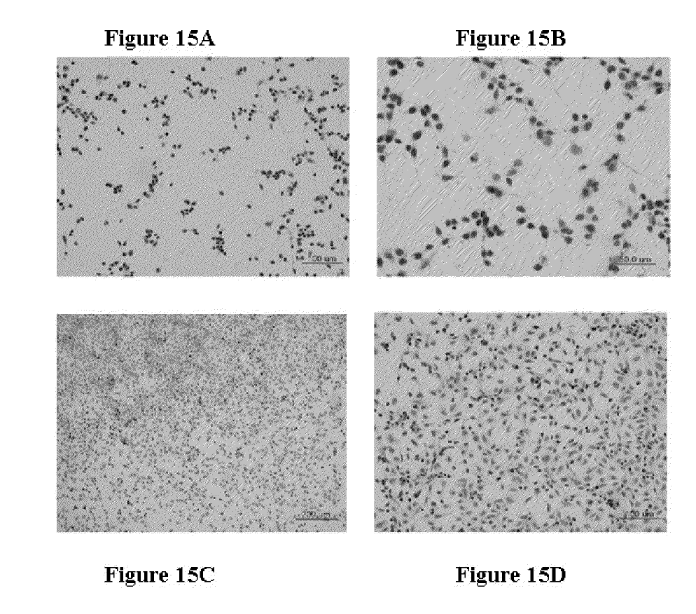

In addition, FIGS. 13 to 15 show the photographs taken at 24, 48 and 72 hours after treatment of cell line HCT-15 with the composition according to the invention or PBS, respectively. It was observed that nuclei of the cancer cells were ruptured in the experimental group, while nuclei and cytoplasm of the cancer cells were developed in the control group.

### Example 6

### Hairs in Nude Mice

It is generally known in the art that the two main defects of mice homozygous for the nude spontaneous mutation (Foxn1^{nu}) are abnormal hair growth and defective development of the thymic epithelium. Although the mice appear hairless, they are born with functional but faulty hair growth follicles. Hair growth cycles and patterns are evident especially in pigmented mice but the faulty follicles do not allow the hair to properly erupt.

During the experiment, the extracts obtained in the Examples 1 to 3 were orally administered to 5-week-old female nude mice (BALB/c nu/nu) at dose level of 0.001 ml/g (volume of extracts/body weight of mouse) twice a day for 2 weeks. The group was consisted of 5 nude mice.

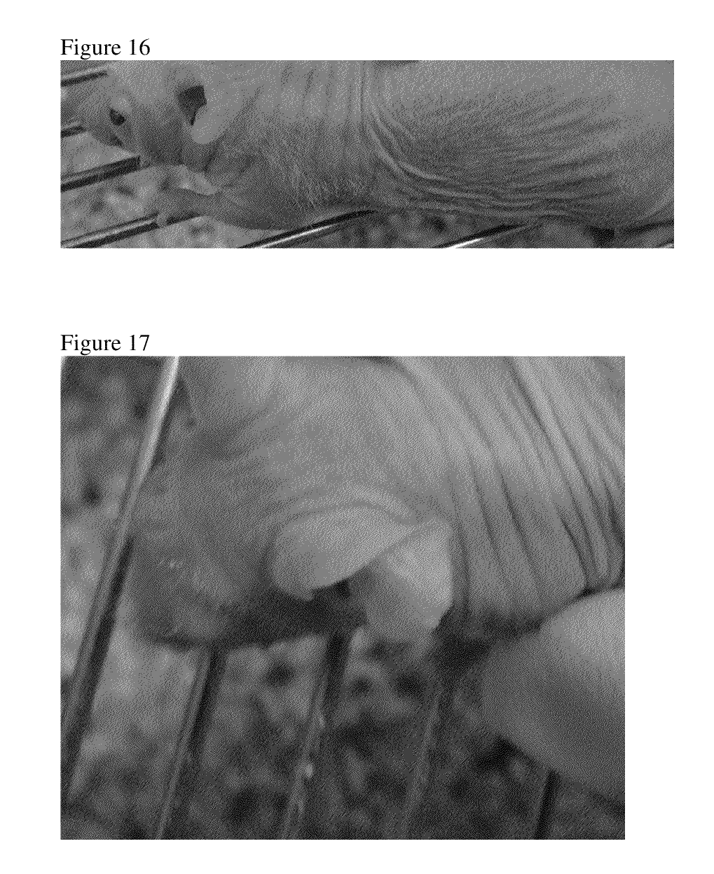

As a result, it is surprisingly found that all of the hairless nude mice are starting to grow hair on their heads and flanks, as time goes by. FIG. 16 shows the photograph taken at 14 days after the extracts of the invention were administered to no. 2 nude mouse, and FIG. 17 shows the photograph taken at 21 days after the extracts of the invention were administered to no. 4 nude mouse.

What is claimed is:

1. An extract or fraction of Cinnamomum aromaticum, Arctium lappa, Viticis fructus, Lonicera japonica, Acanthopanax gracilistylis, Raphanus sativus, Astragalus membranaceus and Hordeum vulgare.

2. A composition for treating or preventing a cancer, comprising the extract or fraction according to claim 1.

3. The composition according to claim 2, wherein said cancer is breast cancer, prostate cancer, lung cancer, liver cancer, pancreatic cancer, skin cancer, colorectal cancer, osteosarcoma or brain tumor.

4. The composition according to claim 2, wherein said Cinnamomum aromaticum, Arctium lappa, Viticis fructus, Lonicera japonica, Acanthopanax gracilistylis, Raphanus sativus, Astragalus membranaceus and Hordeum vulgare are in the ratio of 4˜5:2:4˜7:2˜4:2˜3:2˜8:2:4˜7.

5. The composition according to claim 2, wherein said extract or fraction of Cinnamomum aromaticum comprises 2-methyl-butyric acid.

6. The composition according to claim 2, wherein said extract or fraction of Arctium lappa comprises one or more selected from the group consisting of arctigenin-4-O-glucoside, 3,5,7,9-tetrayne and alanine.

7. The composition according to claim 2, wherein said extract or fraction of Viticis fructus comprises one or more selected from the group consisting of vitexicarpin and hesperidin.

8. The composition according to claim 2, wherein said extract or fraction of Lonicera japonica comprises one or more selected from the group consisting of luteolin-7-rhamnoglucoside, saponin, pinene, trans-geraniol, linalol and chlorogenic acid.

9. The composition according to claim 2, wherein said extract or fraction of Acanthopanax gracilistylis comprises copper.

10. The composition according to claim 2, wherein said extract or fraction of Raphanus sativus comprises sinigrin.

11. The composition according to claim 2, wherein said extract or fraction of Astragalus membranaceus comprises one or more selected from the group consisting of 3′-hydroxyformononetin, kaempferol and water.

12. The composition according to claim 2, wherein said extract or fraction of Hordeum vulgare comprises valine.

13. A composition for preventing loss of hair or stimulating hair growth, comprising the extract or fraction according to claim 1.

14. The composition according to claim 13, wherein said Cinnamomum aromaticum, Arctium lappa, Viticis fructus, Lonicera japonica, Acanthopanax gracilistylis, Raphanus sativus, Astragalus membranaceus and Hordeum vulgare are in the ratio of 4˜5:2:4˜7:2˜4:2˜3:2˜8:2:4˜7.

15. The composition according to claim 13, wherein said extract or fraction of Cinnamomum aromaticum comprises 2-methyl-butyric acid.

16. The composition according to claim 13, wherein said extract or fraction of Arctium lappa comprises one or more selected from the group consisting of arctigenin-4-O-glucoside, 3,5,7,9-tetrayne and alanine.

17. The composition according to claim 13, wherein said extract or fraction of Viticis fructus comprises one or more selected from the group consisting of vitexicarpin and hesperidin.

18. The composition according to claim 13, wherein said extract or fraction of Lonicera japonica comprises one or more selected from the group consisting of luteolin-7-rhamnoglucoside, saponin, pinene, trans-geraniol, linalol and chlorogenic acid.

19. The composition according to claim 13, wherein said extract or fraction of Acanthopanax gracilistylis comprises copper.

20. The composition according to claim 13, wherein said extract or fraction of Raphanus sativus comprises sinigrin.

21. The composition according to claim 13, wherein said extract or fraction of Astragalus membranaceus comprises one or more selected from the group consisting of 3′-hydroxyformononetin, kaempferol and water.

22. The composition according to claim 13, wherein said extract or fraction of Hordeum vulgare comprises valine.

23. A process for preparing the extract according to claim 1, comprising:
a) placing Cinnamomum aromaticum, Arctium lappa, Viticis fructus, Lonicera japonica, Acanthopanax gracilistylis, Raphanus sativus, Astragalus membranaceus and Hordeum vulgare within an extraction apparatus with water, and
b) heating said extraction apparatus to obtain the extracts.

24. The process according to claim 23, wherein water used in the step a) is the water purified by reverse-osmosis membrane filter system.

25. The process according to claim 23, wherein the heating in the step b) is performed at temperature of 100˜120° C. for 4˜5 hours.

26. The process according to claim 23, wherein the extracts obtained in the step b) are filtered by water at 100˜150° C.

27. The process according to claim 26, wherein the filtered extracts are further filtered by water at 70˜100° C.

28. The process according to claim 27, wherein the filtered extracts are further filtered by water at 40˜60° C.

29. The process according to claim 28, wherein the filtered extracts are further filtered by water at 15˜30° C.

30. The process according to claim 29, wherein the filtered extracts are further filtered by water at −1˜15° C.

31. The process according to claim 26, wherein the filter used in said filtering is the membrane with a pore size of not bigger than 10⁻⁶ m.

32. The process according to claim 27, wherein the filter used in said filtering is the membrane with a pore size of not bigger than 10⁻⁶ m.

33. The process according to claim 28, wherein the filter used in said filtering is the membrane with a pore size of not bigger than 10⁻⁶ m.

34. The process according to claim 29, wherein the filter used in said filtering is the membrane with a pore size of not bigger than 10⁻⁶ m.

35. The process according to claim 30, wherein the filter used in said filtering is the membrane with a pore size of not bigger than 10⁻⁶ m.

36. A method of treating or preventing cancer in a human or animal subject, comprising administering to the subject an extract or fraction of claim 1.

37. A method for preventing loss of hair or stimulating hair growth in a human or animal subject, comprising administering to the subject an extract or fraction of claim 1.
